(19) United States
(12) Patent Application Publication
(10) Pub. No.: US 2012/0053609 A1
(43) Pub. Date: Mar. 1, 2012

(54) METHODS FOR FORMING TRACTS IN TISSUE

(75) Inventors: D. Bruce Modesitt, San Carlos, CA (US); Dan J. Hammersmark, San Mateo, CA (US); Joseph F. Paraschac, Campbell, CA (US)

(73) Assignee: ARSTASIS, INC., Redwood City, CA (US)

(21) Appl. No.: 13/289,997

(22) Filed: Nov. 4, 2011

Related U.S. Application Data
(63) Continuation of application No. 11/873,957, filed on Oct. 17, 2007, now abandoned.

(51) Int. Cl. A61B 17/34

(52) U.S. Cl. 606/185

(57) ABSTRACT

Described here are methods for forming one or more tracts in tissue. The tracts may be formed in any suitable or desirable tissue, and may seal relatively quickly without the need for a supplemental closure device. In some variations, the methods comprise advancing a tissue-locating member adjacent to a tissue wall, deforming at least a portion of the tissue wall with the tissue-locating member, and advancing a tissue-piercing member through the deformed tissue to form the tract, where the tract provides access for one or more tools. Also described here are methods of forming tracts through rotated or tented tissue. Any of the methods described here may also be used with tissue having at least one irregular surface.

### RELATED APPLICATION DATA

The present application is a continuation of pending U.S. patent application Ser. No. 11/873,957, filed Oct. 17, 2007, the priority of which is claimed under 35 U.S.C. §120, and the contents of which is incorporated herein by reference in its entirety, as though set forth in full.

### FIELD

Described here are methods for forming tracts in tissue. More specifically, described here are methods for forming tracts in tissue, where at least a portion of the tissue has been deformed.

### BACKGROUND

A number of devices and methods have previously been described for forming tracts in or through tissue. For example, U.S. patent application Ser. Nos. 10/844,247, 11/544,196, 11/545,272, 11/544,365, 11/544,177, 11/544,149, 10/888,682, 11/432,982, 11/544,317, 11/788,509, all of which are incorporated by reference in their entirety herein, describe devices and methods for forming tracts in tissue. In general, the tracts described there self-seal or seal without the need for a supplemental closure device. These tracts may be quite useful in providing access to a tissue location (e.g., an organ lumen) so that one or more tools may be advanced through the tract, and a procedure may be performed. Given the tremendous applicability of such methods, additional methods of forming tracts in tissue would be desirable.

### BRIEF SUMMARY

Described here are methods for forming one or more tracts in tissue. The tracts may be formed in any suitable or desirable tissue. For example, the tissue may be an organ of any of the body systems, (e.g., the cardiovascular system, the digestive system, the respiratory system, the excretory system, the reproductive system, the nervous system, etc.). In some variations, the tissue is an organ of the cardiovascular system, such as the heart or an artery. In other variations, the tissue is an organ of the digestive system, such as the stomach or intestines. The tracts formed here may seal relatively quickly without the need for a supplemental closure device. For example, the tracts may seal within 12 minutes or less, within 9 minutes or less, within 6 minutes or less, within 3 minutes or less, etc. Of course, if necessary or desirable, a supplemental closure device may be used in conjunction with the described methods.

In some variations, the methods comprise advancing a tissue-locating member adjacent to a tissue wall, deforming at least a portion of the tissue wall with the tissue-locating member, and advancing a tissue-piercing member through the deformed tissue to form the tract, wherein the tract provides access for one or more tools. In some variations, deforming at least a portion of the tissue comprises changing the orientation of the tissue wall from a first configuration to a second configuration (e.g., by changing the shape of the tissue wall, etc.). In other variations, deforming at least a portion of the tissue comprises changing the position of the tissue wall (e.g., by rotating or tenting the tissue).

The methods may further comprise deforming at least a portion of the tissue after the tissue-piercing member has been advanced, where deforming the tissue comprises changing the orientation of the tissue wall from a second configuration to a third configuration. A tissue-piercing member may then optionally be advanced through the deformed tissue in the third configuration, affecting needle redirection.

The tract may be of any suitable or desirable length. In some variations, the tissue-piercing member enters the tissue at a first location, and exits the tissue at a second location, and the length between the first location and the second location is greater than the thickness of the tissue wall. In some variations, the length of the tract is greater than the thickness of the tissue wall.

One or more tools may be advanced through the tract, e.g., a wire, a sheath, a catheter, a cutting device, an ablation device, one or more implants, or any other tool.

In some variations, advancement of a tool through the tract increases the diameter, cross-sectional area, perimeter, or general width of the tract.

The methods described here may also be used with tissue having at least one irregular surface. In general, methods of forming a tract in a tissue having at least one irregular tissue surface comprise advancing a tissue-locating member adjacent to a tissue wall, reshaping at least a portion of the tissue wall, wherein the tissue wall has an irregular surface, and advancing a tissue-piercing member through the reshaped tissue to form the tract, where the tract provides access for one or more tools. Similarly, methods for forming tracts in tented or rotated tissue are specifically described. In general, the methods for forming a tract in tented tissue comprise advancing a tissue-locating member adjacent to a tissue wall, tenting at least a portion of the tissue wall, and advancing a tissue-piercing member through the tented tissue to form the tract, wherein the tenting immobilizes at least a portion of the tissue wall during advancement of the tissue-piercing member therethrough.

The methods for forming a tract in rotated tissue generally comprise advancing a tissue-locating member adjacent to a tissue wall, rotating at least a portion of the tissue wall, and advancing a tissue-piercing member through the rotated tissue to form the tract, where the rotating helps position the tissue-piercing member relative to the tissue wall. Any of the methods described here may also comprise immobilizing at least a portion of the tissue.

### BRIEF DESCRIPTION OF THE DRAWINGS

FIGS. 1-3 show a standard Seldinger procedure for placement of a wire through a tissue.

FIG. 4 shows one variation of a device that may be used with the methods described herein.

FIGS. 5-9 show how the device of FIG. 4 may be used to access a lumen of a tissue.

FIG. 10 provides a close up view of a distal portion of the device of FIG. 4.

FIG. 11 shows a tissue-locating member sprung upward after an articulation section has been released.

FIG. 12 shows a tissue-locating member contacting the inner surface of a lumen wall.

FIG. 13 shows advancement of a tissue-piercing member into a lumen wall.

FIG. 14 shows further tenting of the tissue wall and redirection of a tissue-piercing member, from a first direction to a second direction, or from a second direction to a third direction, as the case may be.

FIG. 15 shows a tissue-piercing member being further advanced in the lumen wall, and through the lumen wall until it enters the tissue lumen.

FIGS. 16-17 show advancement of a wire through the tissue-piercing member.

FIG. 18 shows the wire in the lumen after the device has been proximally withdrawn.

FIG. 19 shows advancement of a sheath over the wire for introduction of one or more tools therethrough.

FIG. 20 shows the sheath in the tissue lumen after a dilator has been withdrawn.

FIG. 21 shows the tract formed in the tissue, after the device, and any additional tools have been withdrawn.

FIG. 22 shows a cross-sectional view taken along line A-A of FIG. 13.

FIG. 23 shows the relative positions of a tissue-piercing member and a tissue-locating member prior to rotation.

FIG. 24 shows a cross-sectional view of the tissue of FIG. 22 with some tenting or tissue deformation.

FIGS. 25-27 illustrate how the methods described here may be used to form tracts through one or more irregular tissue surfaces.

### DETAILED DESCRIPTION

Described here are methods for forming tracts in tissue, and in general, the tracts formed by the methods described here seal relatively quickly without the need for a supplemental closure device. In some variations the methods comprise forming a tract in tissue by advancing a tissue-locating member adjacent a tissue wall, deforming at least a portion of the tissue wall with the tissue-locating member, and advancing a tissue-piercing member through the deformed tissue to form the tract, where the tract provides access for one or more tools. Deforming at least a portion of tissue may comprise changing the tissue wall from a first configuration to a second configuration, e.g., by changing the shape, position, etc. In some variations, deforming at least a portion of tissue comprises rotating the tissue. In other variations, deforming at least a portion of tissue comprises tenting the tissue. At least a portion of the tissue may be immobilized during the formation of a tract, as will be described in more detail below.

It should be understood that the methods described here may be used with any desirable tissue. For example, the tissue may be an organ, such as an organ of any of the body systems (e.g., cardiovascular system, respiratory system, excretory system, digestive system, reproductive system, nervous system, etc.). In some variations, the methods are used with tissue of the cardiovascular system, such as the vasculature or the heart. In some variations, the tissue is an artery, and the methods are used in conjunction with performing an arterial puncture. In other variations, the tissue is an organ of the digestive system, such as the stomach, or intestines.

### I. Methods of Forming Tracts in Tissue

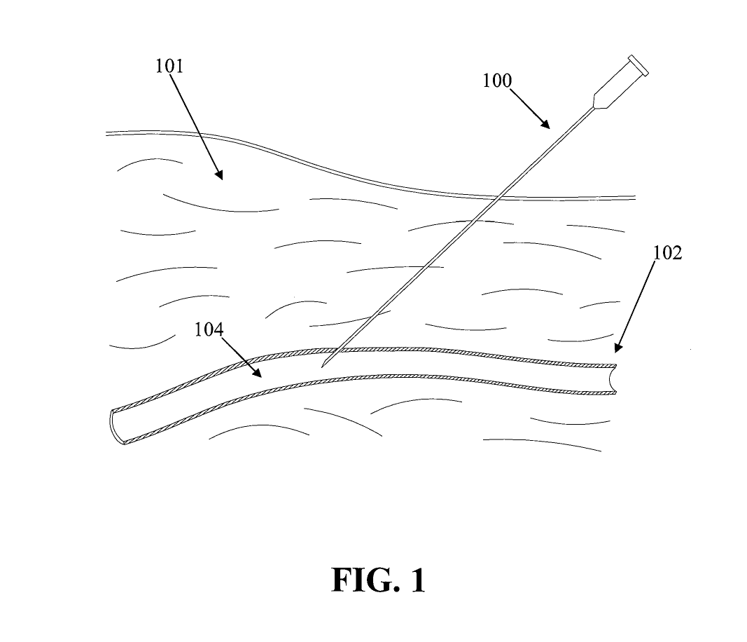

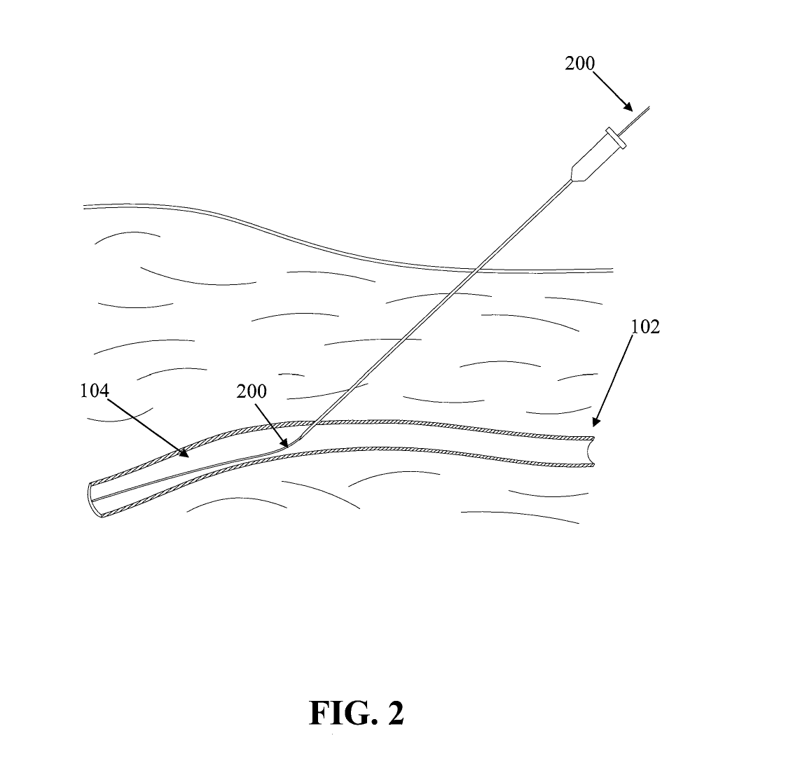

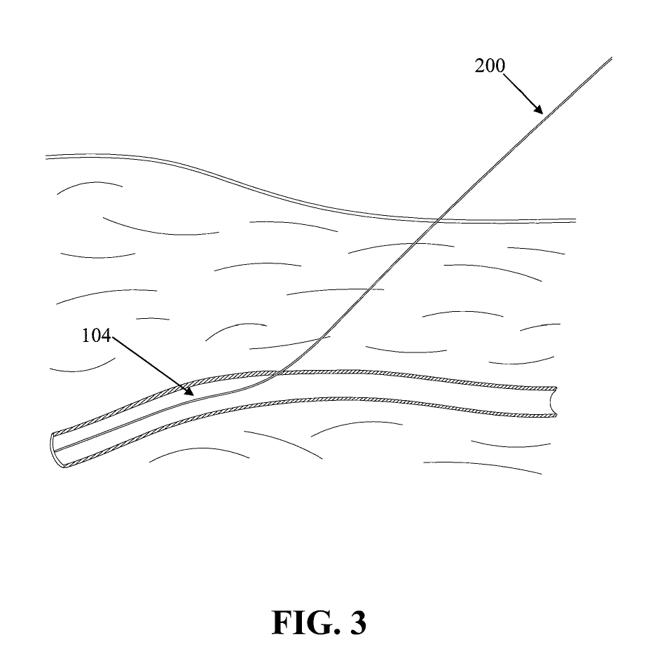

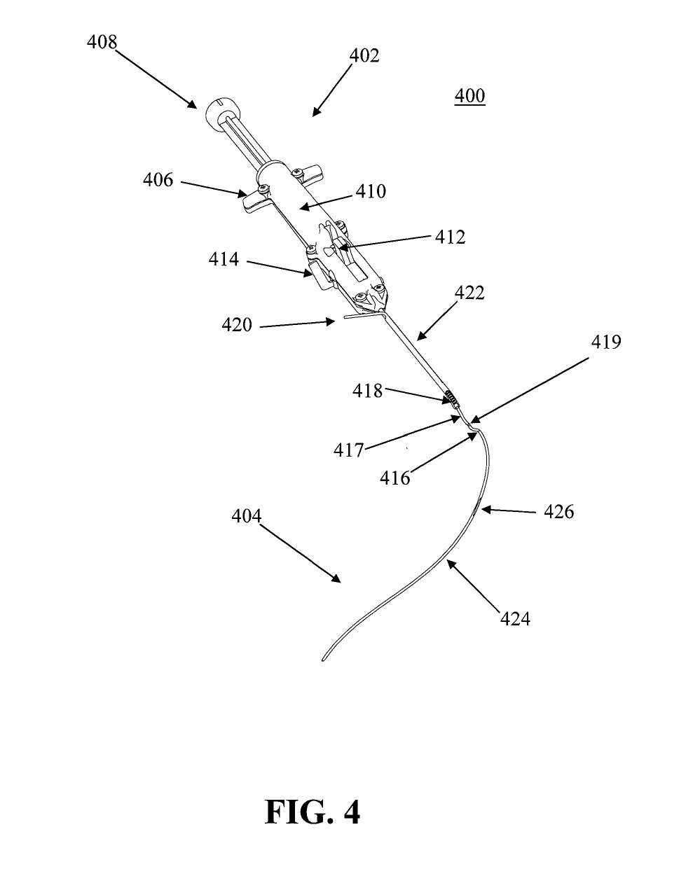

Shown in FIGS. 1-3 is a standard Seldinger procedure for placement of a wire through a tissue. Shown in FIG. 1, for example, is advancement of a needle (100) through subcutaneous tissue (101) into artery (102). Entry into lumen (104) by the needle (100) may optionally be visually confirmed by observing a flash of blood (i.e., blood flow) through the needle. FIG. 2 shows advancement of a wire (200) through needle (100) and into the lumen (104) of the artery (102). After placement of the wire (200), the needle may be withdrawn proximally, leaving the wire (200) in the lumen (104) as shown in FIG. 3. FIG. 4 illustrates one variation of a device that may be used to form tracts in tissue in accordance with the various methods described here. Shown there is device (400), having a proximal portion (402), for use outside the body tissue, and a distal portion (404). Proximal portion (402) of device (400) comprises handle (406), plunger (408), and main body (410). Main body (410) comprises an actuator (412) and one or more levers (414).

In the variation shown in FIG. 4, actuator (412) is used to deploy a retainer (not shown in this figure) and to cause articulation of a tissue-locating member (416) at an articulation section (418) via a hinge-like mechanism. This articulation may be affected in any number of ways. For example, the articulation section (418) may be comprised of one or more shape memory materials, spring members, or combinations thereof. In the variation shown in FIG. 4, the articulation section (418) is comprised of one or more counter-opposed stainless steel slotted tubes having a nickel titanium needle therethrough. In this variation, a spring (not shown) in main body (410) aids axial motion of the counter-opposed slotted tubes, causing flexure of the articulation section (418).

The device may have one or more levers for any suitable purpose. For example, the lever (414) shown in FIG. 4 may be used to aid in the actuation described above by causing additional articulation at the articulation section (418). The lever (414) may also optionally be used to lock the tissue-locating member (416) in the articulated position (or in intermediate articulated positions). The device may also comprise additional levers to lock the tissue-locating member (416) in intermediate articulated positions. Of course, the device need not have any levers, for example, in the case where the tissue-locating member (416) is locked (or unlocked) automatically by the actuation member (412).

Also shown in FIG. 4 is marker port (420), which may be used to indicate that the device has been properly inserted into a lumen by allowing a flash of blood to flow therethrough. In variations where no marker port (420) is used, a flash of blood may be visualized through an opening in the plunger (408). The handle (406) shown in FIG. 4 is configured to facilitate easy use of plunger (408), actuating member (412), and lever (414). Of course, additional configurations of handle (406) may be used as well.

Also shown in FIG. 4 is a needle guide (417), having an opening therein (419), for a needle to exit therethrough. Deployment of a needle using device (400) will be discussed in more detail below. Shaft (422) and guide cannula (424) having a side port (426) therein are also shown in FIG. 4, and will be described in more detail below with reference to the methods.

Any suitable materials for the device (400) may be used. For example, it may be comprised of one or more biocompatible plastic materials (e.g., an injection molded polycarbonate), stainless steels, shape memory materials, combinations thereof, or any other suitable materials. Other suitable devices for use with the methods described herein are disclosed in detail in Applicant's previous applications, which were incorporated by reference herein, above.

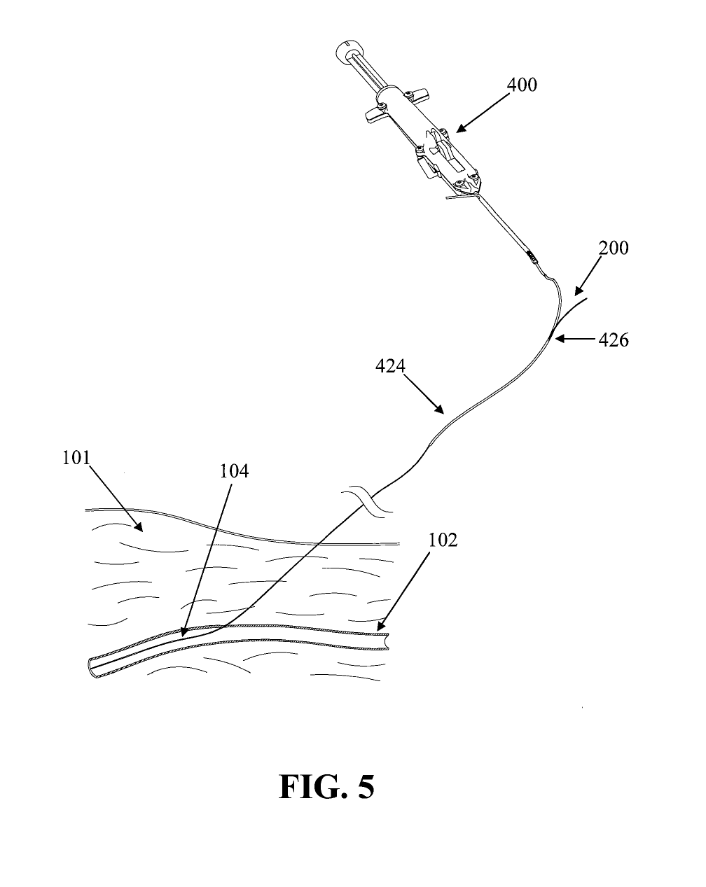

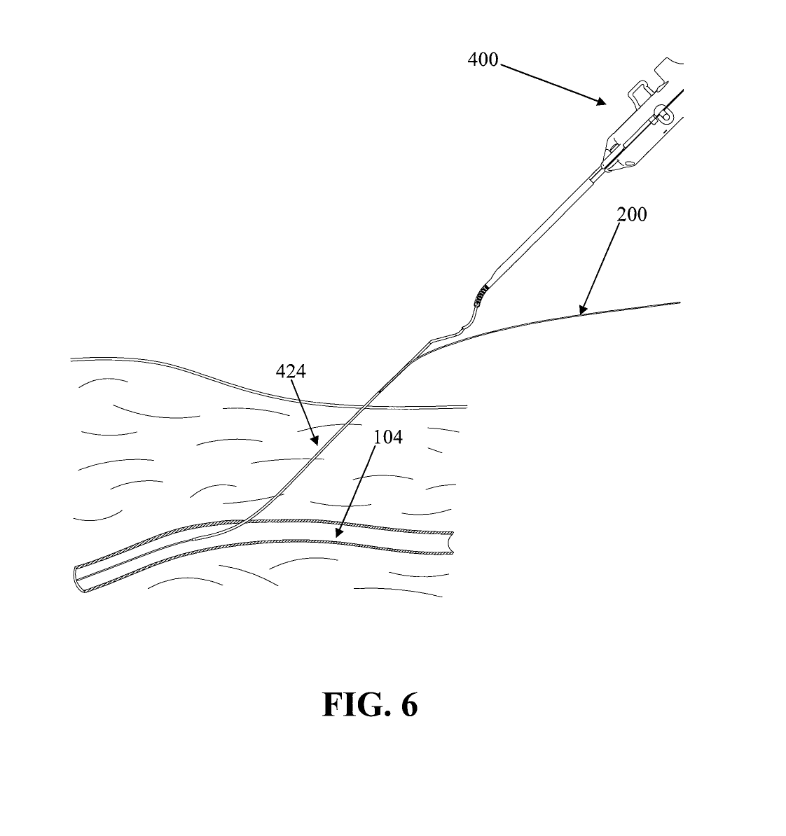

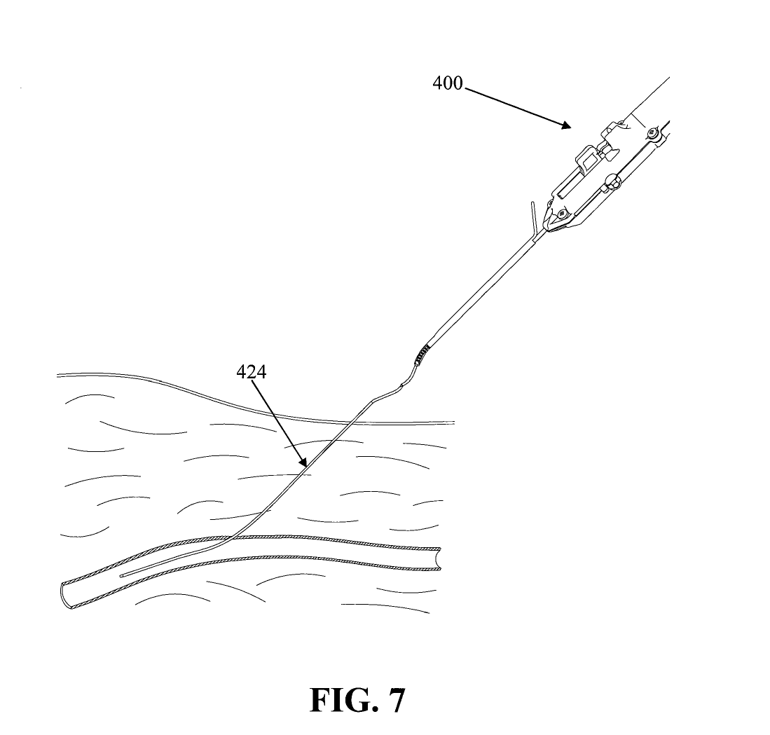

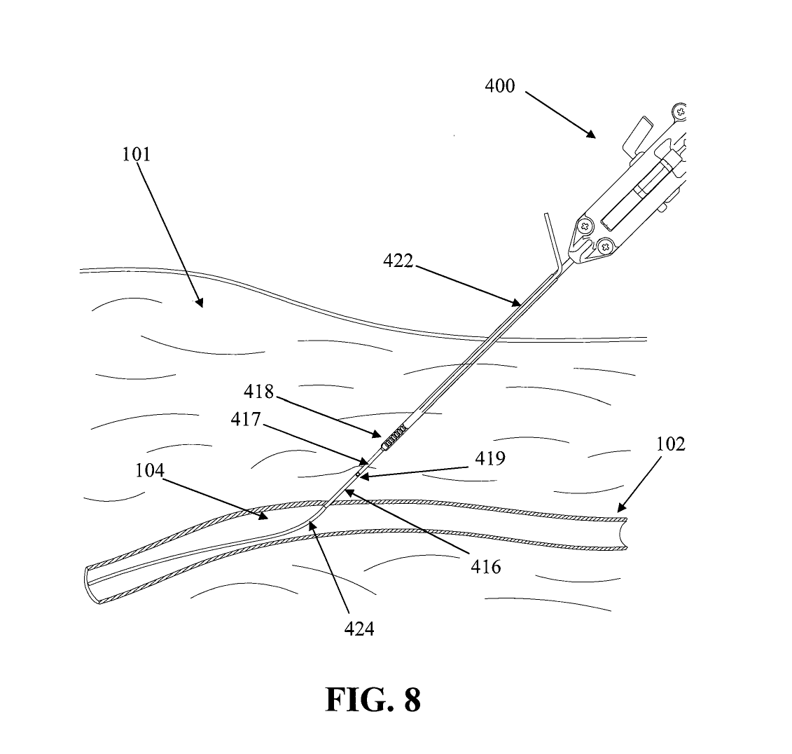

FIGS. 5-9 show how the device of FIG. 4 may be used to access a lumen of a tissue. Shown there is device (400) being inserted through subcutaneous tissue (101) and into the lumen (104) of an artery (102). Of course, while the methods described here are shown with specific reference to an artery, it should be understood, that as described above, the methods may be used with any suitable tissue. Shown in FIG. 5, is insertion of wire (200) (placed previously using a standard Seldinger procedure as described above with reference to FIGS. 1-3) through guide cannula (424). As shown there, wire (200) exits a side port (426). FIG. 6, shows advancement of guide cannula (424) into lumen (104) using wire (200) as a guide for placement (i.e., the guide cannula is advanced over the wire and into the lumen). After placement of the guide cannula (424) into lumen (104), the wire (200) may be removed, as shown in FIG. 7. As will be described in more detail below, the device (400) may also be rotated during insertion. The device (400) shown in FIG. 7 has been, for example, rotated 45 degrees, and the device (400) shown in FIG. 8 has been rotated a further 45 degrees (90 degrees total). Of course, as will be described below, any degree of rotation, in either direction, may be used as desirable.

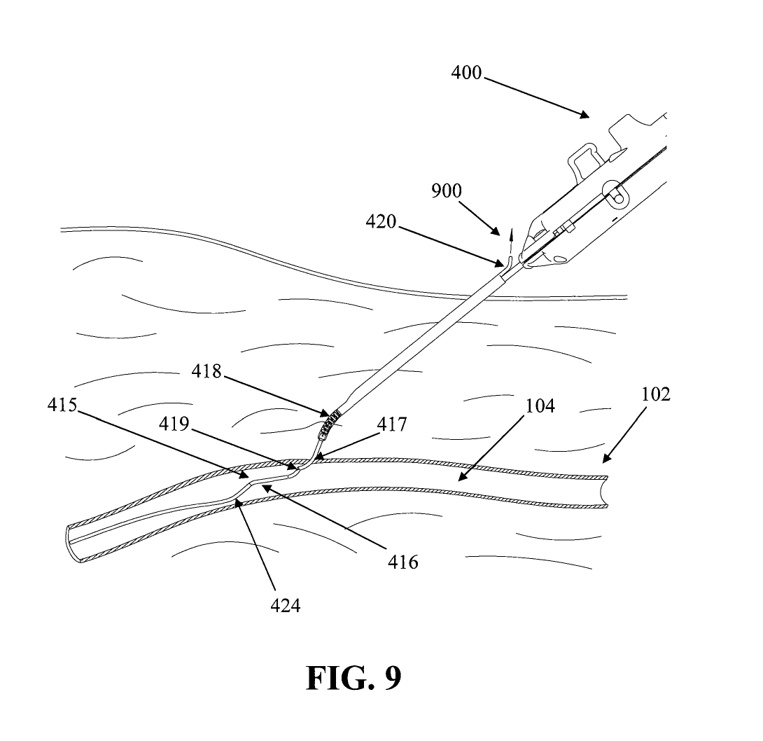

FIG. 8 illustrates further advancement of device (400) into tissue. As shown there, device (400) has been advanced so that the shaft (422), articulation section (418) and needle guide (417) have entered subcutaneous tissue (101). The tissue-locating member (416) has been advanced through subcutaneous tissue (101) and is beginning to enter the lumen (104) of artery (102). Guide cannula (424) is also shown in lumen (104). FIG. 9 shows device (400) where needle port (419) has been advanced into the lumen, verified by visualizing a flash of blood (900) out of marker port (420). At this position, tissue-locating member (416) has been advanced so that it fully resides within lumen (104) of artery (102). In this variation, the tissue-locating member (416) has a titled nose (415) at its distal end that helps bias the tissue-locating member (416) towards the center of the lumen (104). Of course, the tissue-locating member need not have a titled nose (415) as shown.

The distal end of needle guide (417) has been advanced slightly into the lumen (104) to expose opening (419) to blood flowing through lumen (104). The needle guide (417) is in fluid communication with the marker port (420), so that blood entering opening (419) may exit through marker port (420) indicating that the needle guide has been correctly positioned in the lumen (104) as noted above (and shown by 900). FIG. 9 also shows how the device (400) has been rotated back 90 degrees, to the original advancement position shown in FIG. 6). Articulation section (418) is shown slightly flexed. In this configuration, the articulation section (418) is locked via a latch mechanism (not shown) in the main body (410) in an insertion position.

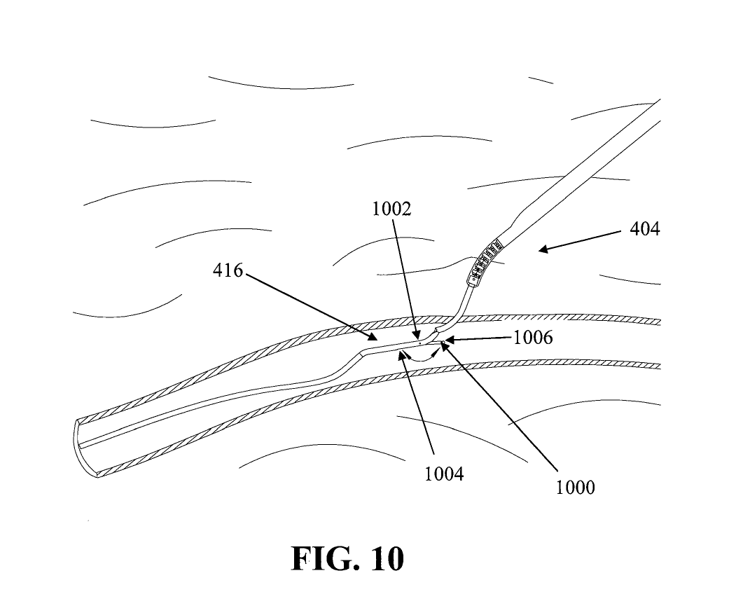

FIG. 10 provides a close up view of distal portion (404) of device (400). In this figure, a retainer (1000) has been deployed from a retainer opening (1004) in the tissue-locating member (416). As described in any of Applicant's previous applications incorporated by reference above, for example, a retainer may be useful for aiding in the positioning of the tissue-locating member (416). Here, the retainer has been deployed via actuator (412) swinging outwardly about retainer pivot (1002) from the retainer opening (1004), here, shown as a slot within tissue-locating member (416). While the retainer shown in FIG. 10 is a hypotube connected to actuator via a wire (not shown), other retainers may be used. Also shown in FIG. 10 is latch (1006), that may be used to maintain the retainer in the retainer opening in its undeployed position when desirable. In this configuration, the latch (1006) is a spherical member configured to be captured by a portion of the retainer opening (1004).

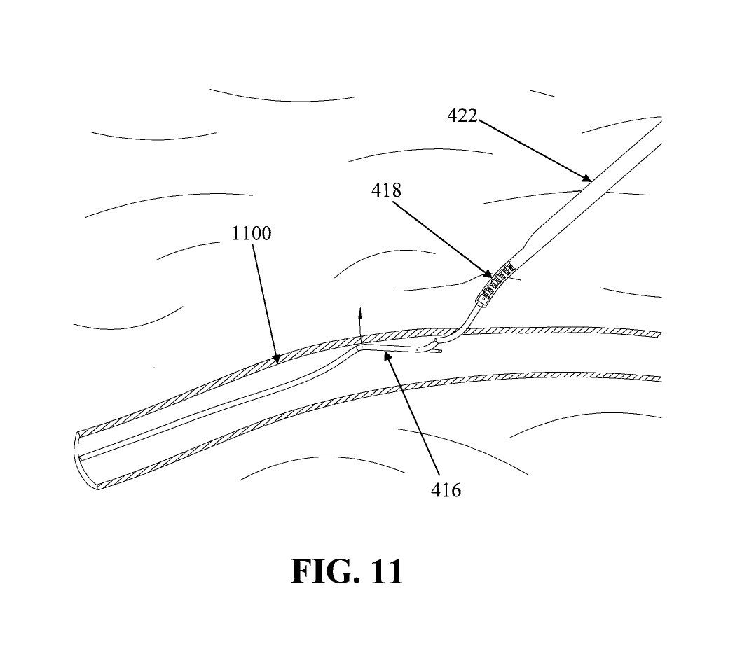

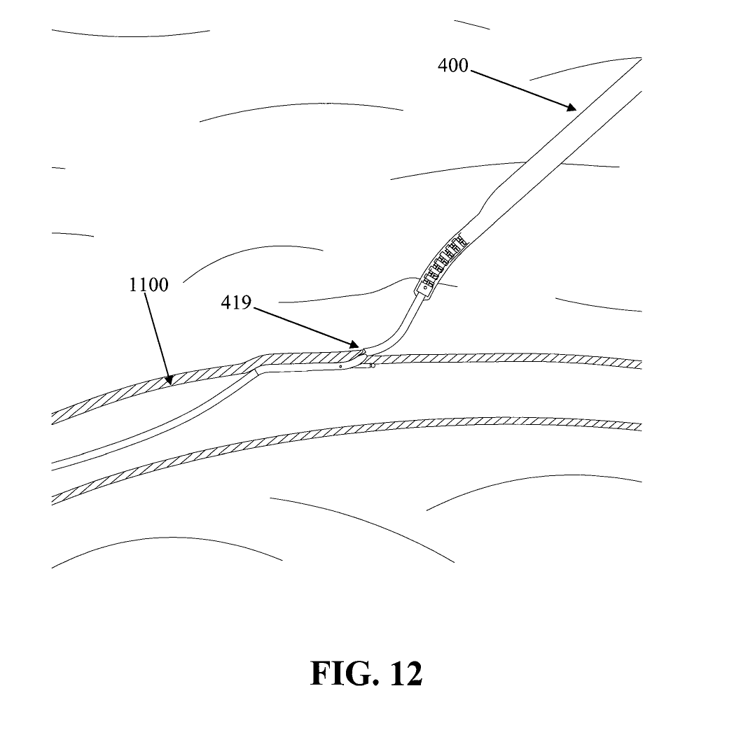

FIG. 11 shows the tissue-locating member (416) after the articulation section (418) has been released, causing the tissue-locating member (416) to spring upward (as shown by the arrow). In this figure, at least a portion of the tissue-locating member (416) is in contact with the lumen wall (1100), however, this need not be so. Indeed, whether a portion of the tissue-locating member (416) contacts the lumen wall (1100) is largely dictated by the position of shaft (422) (i.e., how far the device has been advanced into the tissue). After the articulation member (418) is released, the device (400) is pulled proximally, so that the tissue-locating member (416) contacts the inner surface of the lumen wall (1100) as shown in FIG. 12. Also shown here is opening (419) within the tissue wall. In this position, no blood will flow through the opening to the marker port, thus providing a visual indication that the needle guide is no longer in the lumen (104). In this way, proper positioning of the device may be facilitated. As the tissue-locating member (416) contacts the inner surface of lumen wall (1100) it deforms at least a portion of the tissue, causing it to tent slightly. In this variation, the tissue-locating member effectively immobilizes a portion of the tissue, in preparation for advancing a tissue-piercing member therethrough.

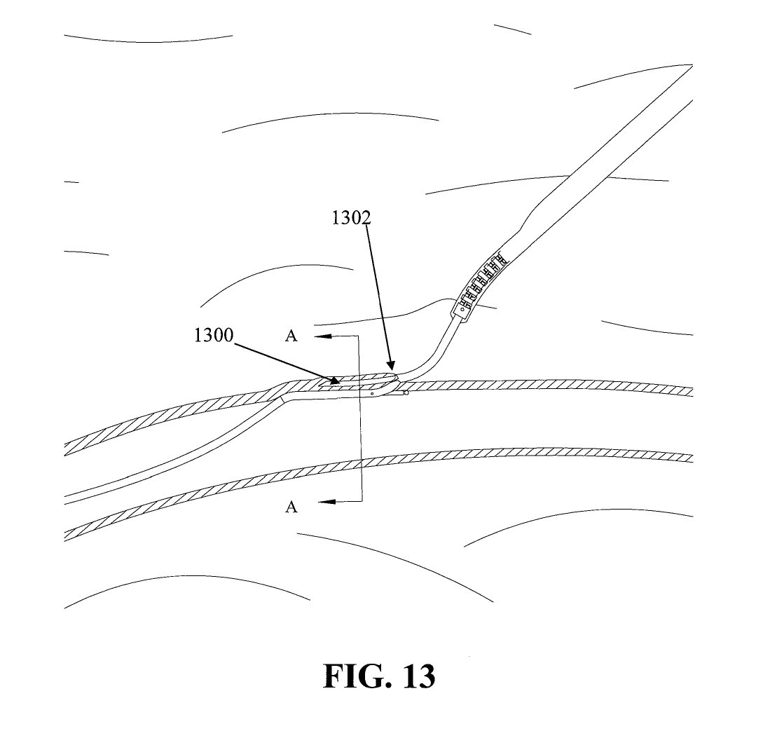

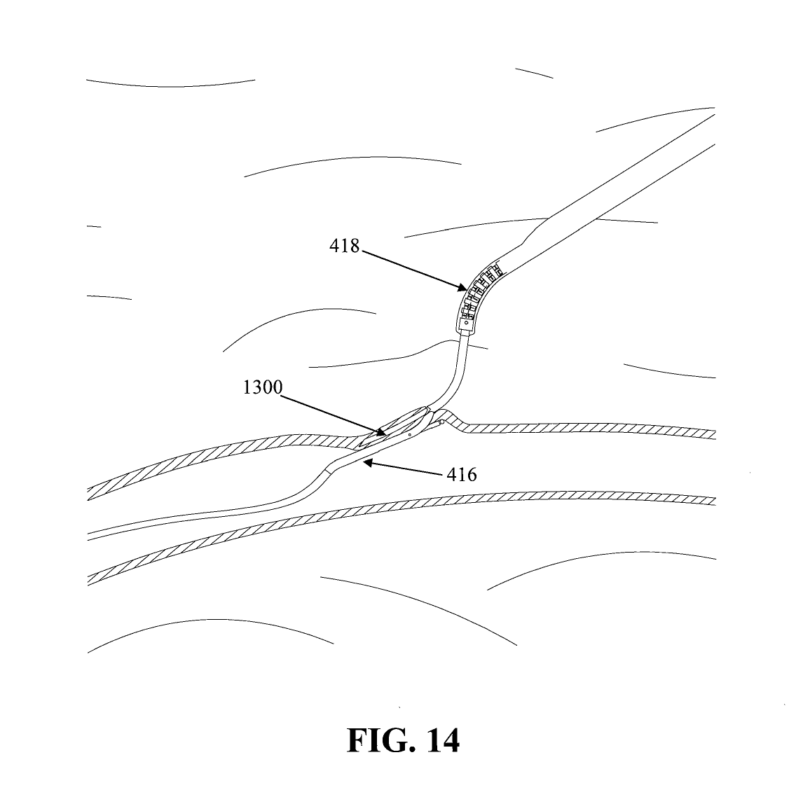

FIG. 13 shows advancement of a tissue-piercing member (1300) into the lumen wall (1100), here, entering the lumen wall at a first location (1302) and being advanced laterally into the lumen wall. Notably, the tissue-piercing member (1300) shown here has a slight curve, indicating that the tissue-piercing member (1300) need not be straight. After the tissue-piercing member (1300) has been advanced into the lumen wall (1100) as shown in FIG. 13, the articulation section (418) may be flexed from its released position to a pitched forward position, causing the tissue-locating member (416) to further tent the tissue, and causing the tissue-piercing member (1300) to be redirected from a first direction to a second direction, or from a second direction to a third direction, as the case may be, as shown in FIG. 14.

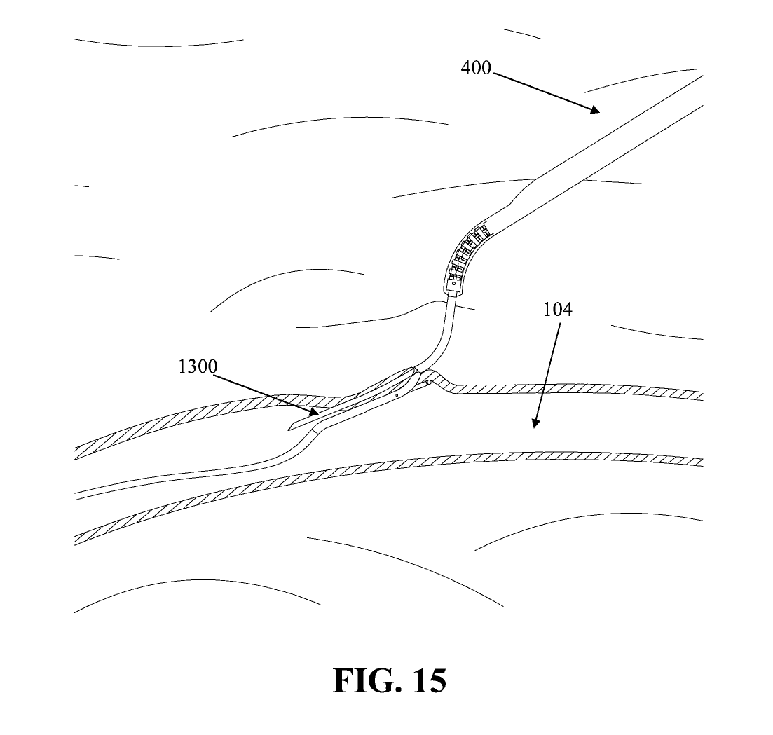

FIG. 15 shows the tissue-piercing member (1300) being further advanced in the lumen wall, and through the lumen wall (1100) until it enters lumen (104). As the tissue-piercing member (1300) is advanced into the lumen (104), a flash of blood may be visualized, either through a marker port, or through an opening in the plunger, as described above. In this way, proper positioning of the tissue-piercing member (1300) within the lumen may be confirmed. If further advancement of the tissue-piercing member (1300) does not result in entry in the lumen (e.g., if calcification prevents proper needle redirection, or if there is unfavorable anatomy or device positioning, etc.), the device (400) may be withdrawn proximally until the guide cannula side port (426) is exposed outside the body. At this point a decision may be made to try with another device, or to use a standard arteriotomy procedure (in the case where the tissue is an artery).

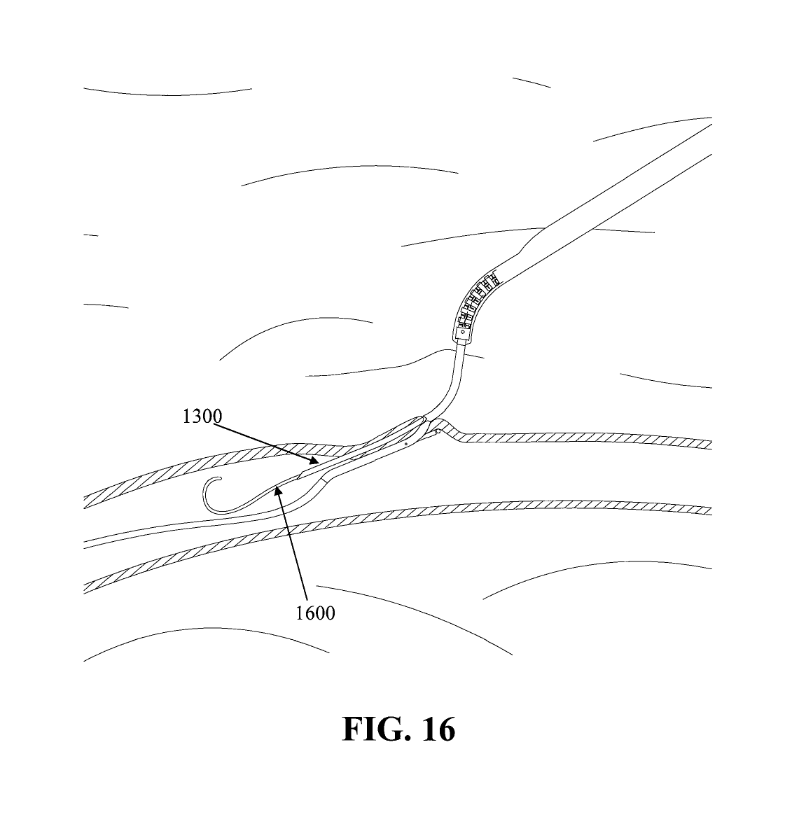

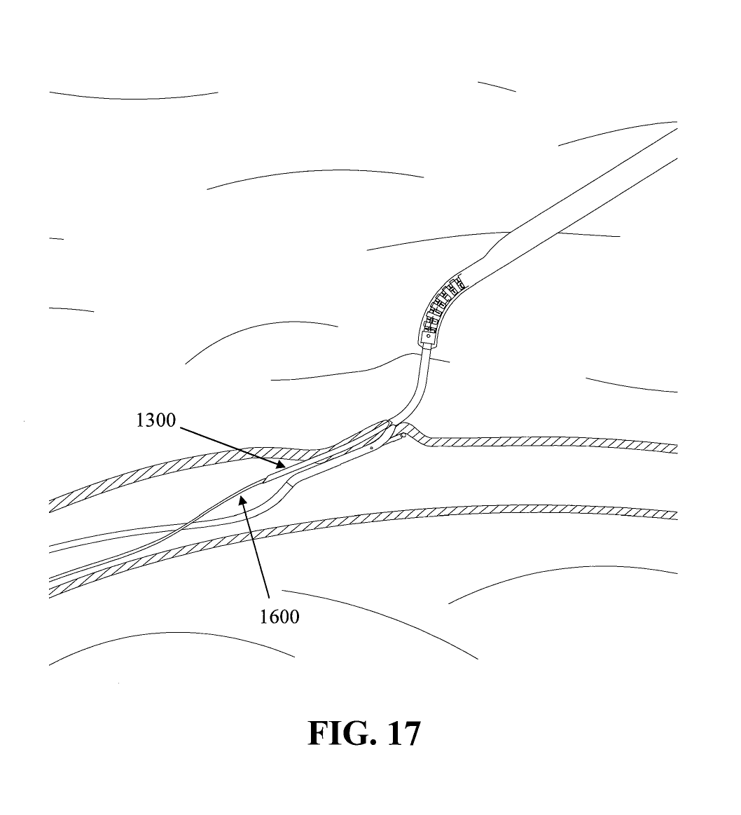

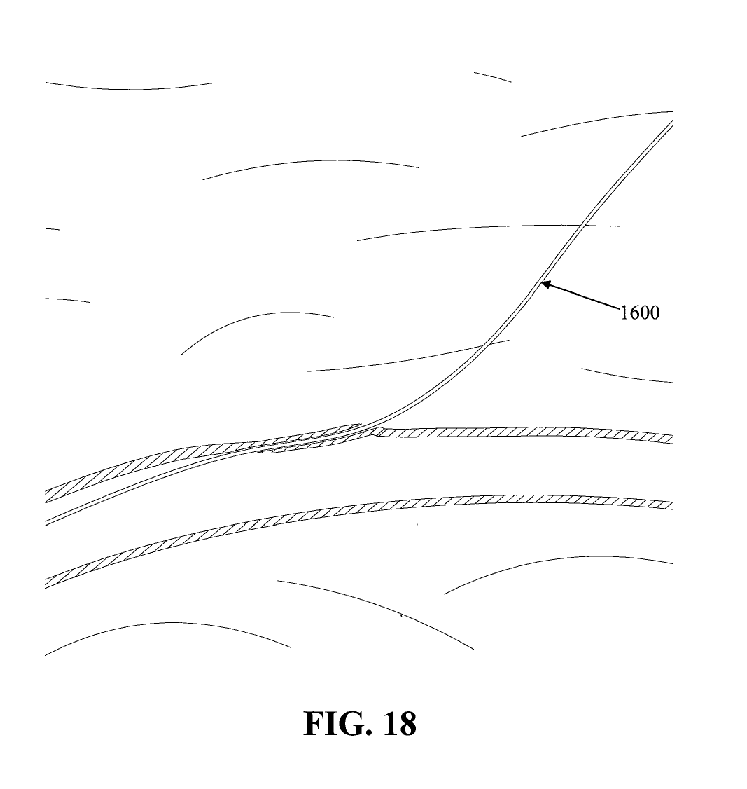

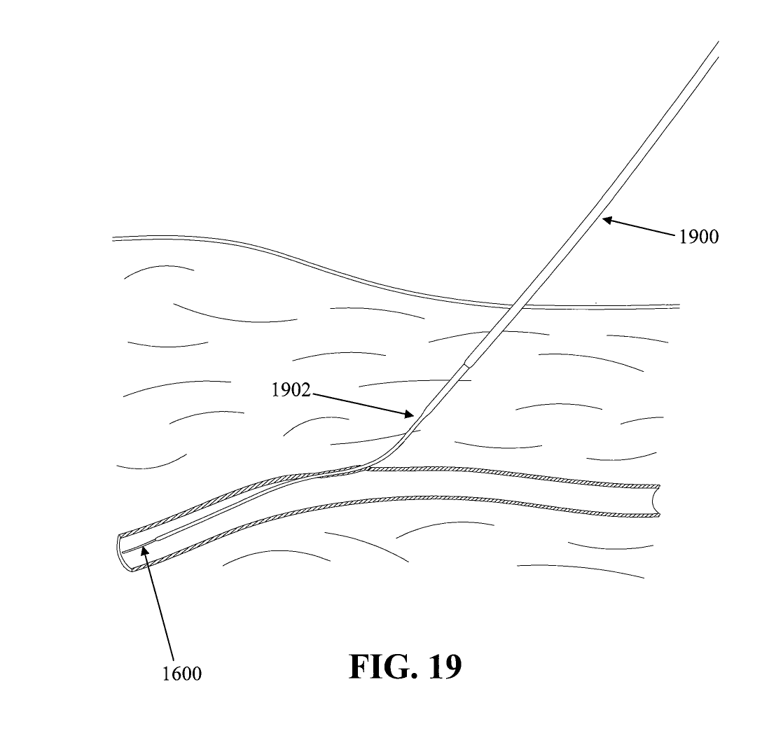

FIGS. 16 and 17 show advancement of a wire (1600) through the tissue-piercing member (1300). The wire (1600) may be the same wire as wire (200) described above, or may be a different wire. The wire will then act as a guide for advancement of one or more tools into the lumen after the device has been proximally withdrawn as shown by FIG. 18. For example, FIG. 19 shows advancement of a sheath (1900) over wire (1600) for introduction of one or more tools therethrough.

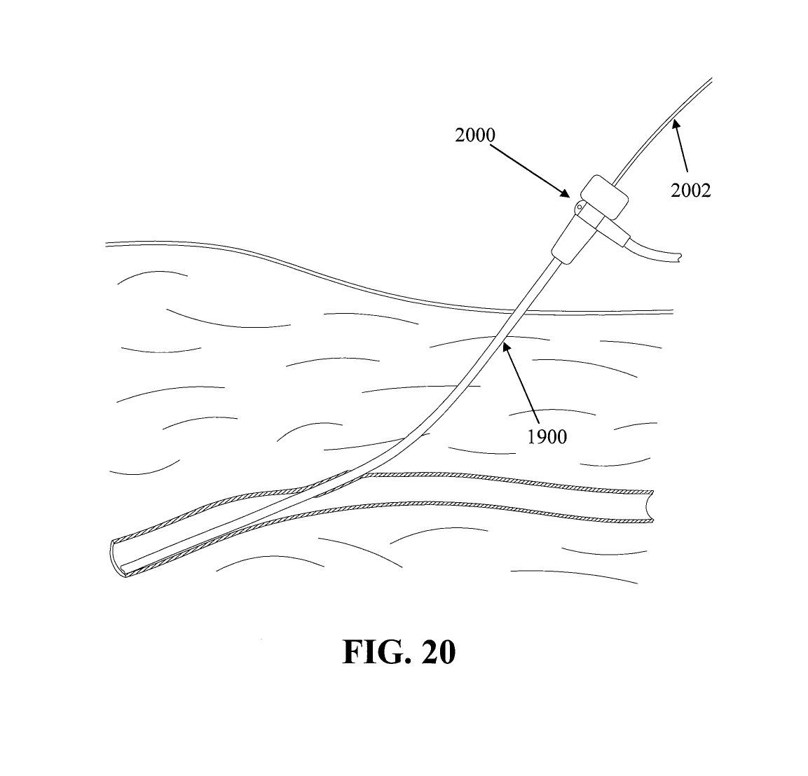

As shown in FIG. 19, the sheath (1900) is slidably coupled to a dilator (1902). The dilator may be advanced through the lumen of sheath (1900), and be used to facilitate positioning of the sheath in the lumen (104) of the artery (or other tissue as the case may be). As shown in FIG. 19, the dilator (1902) has an elongated tip, having a distal cross-sectional diameter smaller than the cross-sectional diameter near its proximal end. This type of sheath/dilator system may be particularly advantageous if the sheath (1900) has a much greater cross-sectional diameter (e.g., 5 F-12 F) than the wire (1600) (e.g., 0.012 to 0.35 inches) over which it will be advanced, since the wire (1600) may not provide sufficient structural support for insertion of the sheath. Here, the end of dilator (1902) having a smaller cross-sectional diameter is more easily advanced over the wire (1600) and thus provides better support for the larger diameter portions to follow. In this way, the cross-sectional area of the tract is gradually increased, which may help in reducing trauma to the tissue. FIG. 20 shows the sheath (1900) in the lumen after the dilator (1902) has been withdrawn. Also shown is the proximal end (2000) of the sheath having an opening therein for introduction of one or more tools (2002).

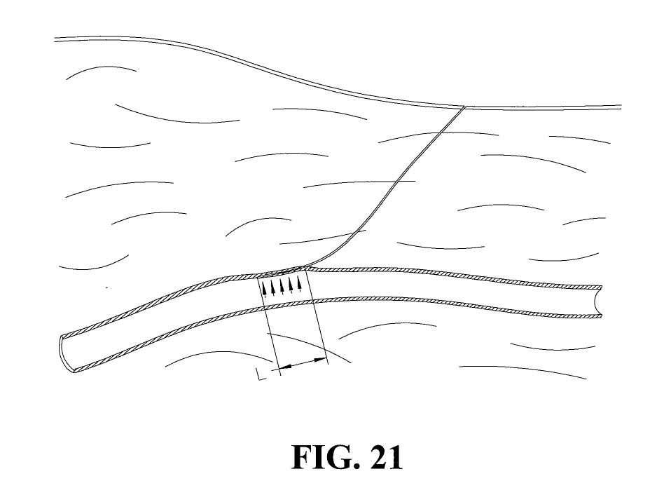

FIG. 21 shows the tract (2100) formed in the tissue, after the device, and any additional tools have been withdrawn. If desirable, a filament (e.g., a wire, a polymer, etc.) or suture material having any suitable cross-section may be left in the tract and exit the body. In this way, if re-access to the lumen is desirable (for example, for placement of a supplemental closure device, for performing additional procedures, etc.) the filament or suture may be used as a guide over which re-access may be accomplished using one or more tools.

As shown in FIG. 21, the tract is generally diagonal, and has a length L. The length may be any suitable or desirable length to help facilitate relatively rapid sealing of the tract. For example, when the methods described here are used with the vasculature, a longer tract may be desirable. This is thought to be because a longer tract will expose helpful biological factors (e.g., growth factors, etc.) that will aid in the sealing (this may also be the case with other tissue as well). In addition, a longer tract will have a larger area for mechanical pressure to act on, sealing the tract more quickly. In some variations, the length is greater than the thickness of the lumen wall (1100). The arrows shown in FIG. 21 illustrate how pressure acting on the tract causes the tract to seal relatively rapidly without the need for an additional closure device. For example, the tract may seal in 12 minutes or less, 9 minutes or less, 6 minutes or less, 3 minutes or less, etc, reducing the duration of any external compression that may be needed. Of course, if desirable, an additional closure device (e.g., plug, clip, glue, suture, etc.) may be used.

### Rotating

Also described here are methods of forming tracts in rotated tissue. In some variations, these methods comprise, advancing a tissue-locating member adjacent to a tissue wall, rotating at least a portion of the tissue wall, and advancing a tissue-piercing member through the rotated tissue to form the tract, wherein the rotating helps to position the tissue-piercing member relative to the tissue wall. Tissue rotation may be particularly desirable, e.g., when the initial Seldinger stick is performed off the center-axis.

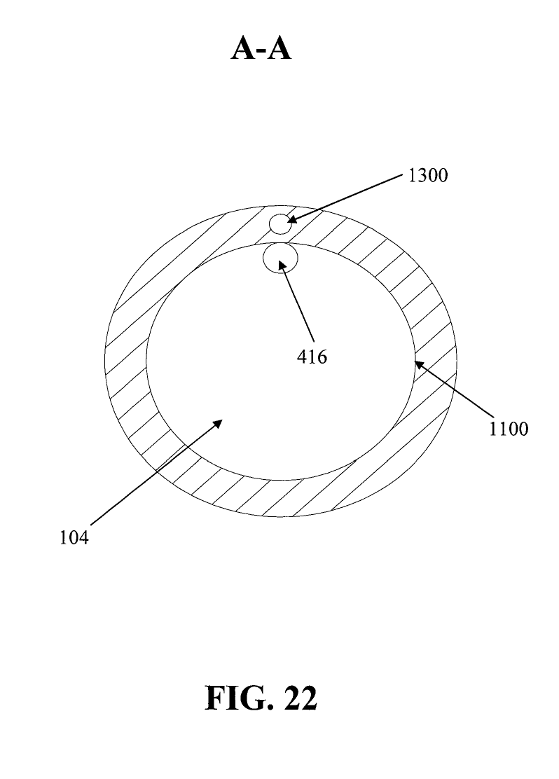

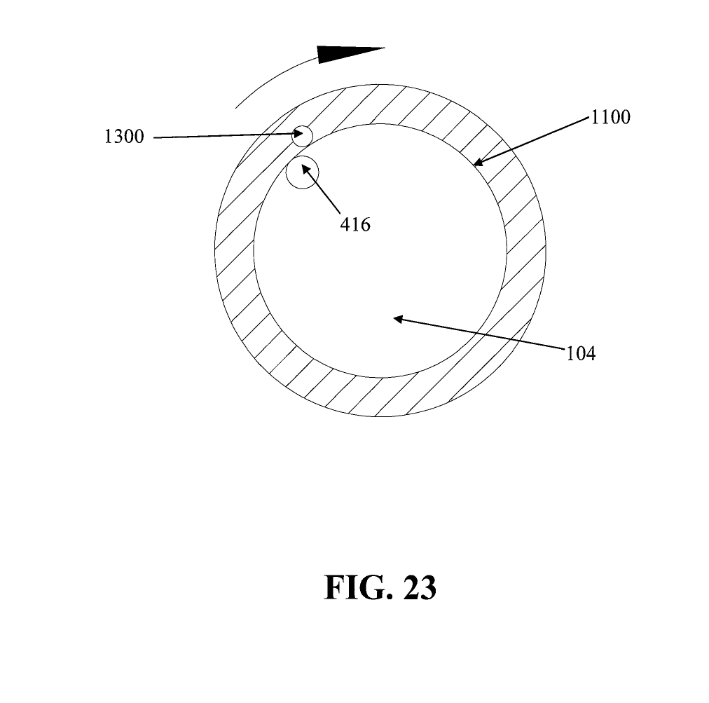

FIG. 22 shows a cross-sectional view taken along line A-A of FIG. 13. Shown there is tissue-piercing member (1300) inside tissue wall (1100). Tissue-locating member (416) is shown within the lumen (104), immediately adjacent to, and contacting inner surface of tissue wall (1100). As shown in this figure, the tissue has been rotated so that the tissue-piercing member (1300) is desirably positioned in the tissue wall, and away from one of the tissue wall surfaces. For example, FIG. 23 shows the relative positions of tissue-piercing member (1300) and tissue-locating member (416) prior to rotation (shown by arrow).

In this way, rotation of the tissue may be useful to effect a desirable tissue-piercing member location, which may in turn be useful for forming a tract having suitable thicknesses of tissue on either side. This may help ensure that the tract is robust enough to withstand repetitive insertion of various tools. In addition, having sufficient tissue thickness on either side of the tract may help the tract seal more quickly. Initial positioning of the tissue-piercing member away from one or more surfaces of the tissue wall may also help with the formation of a longer tract, which may also be useful in ensuring more rapid sealing.

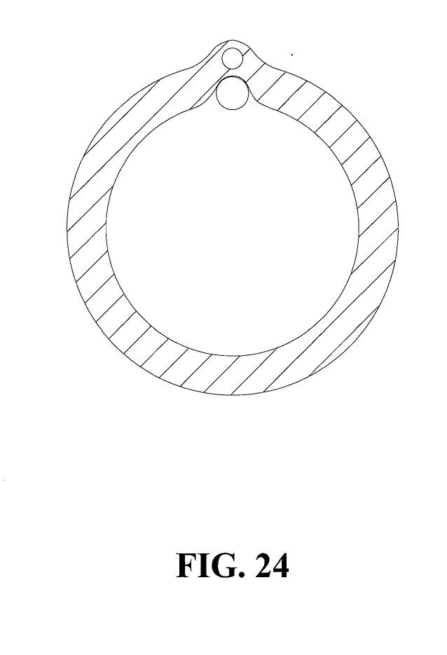

Of course, rotation of the tissue may be used in combination with any other method of tissue deformation described herein. For example, FIG. 24 shows a cross-sectional view of the tissue of FIG. 22 with some tenting or tissue deformation. It should be understood that the tenting or deformation need not occur as a separate step. Indeed, rotation and deformation may be performed concurrently.

The tissue may be rotated in either direction about a tissue circumference (e.g., from 0.degree.-360.degree., from 0.degree.-180.degree., from 0.degree.-45.degree., from 45.degree.-90.degree., etc.). However, the tissue need not be rotated a significant amount (e.g., the tissue may be rotated 1.degree., 5.degree., 10.degree., 15.degree., etc.) and the entire tissue thickness need not be rotated. For example, in some instances it may be desirable to rotate only the tissue nearest the tissue-locating member, etc.

### Forming Tracts in Tissue Having an Irregular Tissue Surface

Also described here are methods for forming tracts in a tissue having an irregular tissue surface. In some variations, the methods comprise advancing a tissue-locating member adjacent a tissue wall, reshaping at least a portion of the tissue wall, wherein the tissue wall has an irregular surface, and advancing a tissue-piercing member through the reshaped tissue to form the tract, where the tract provides access for one or more tools.

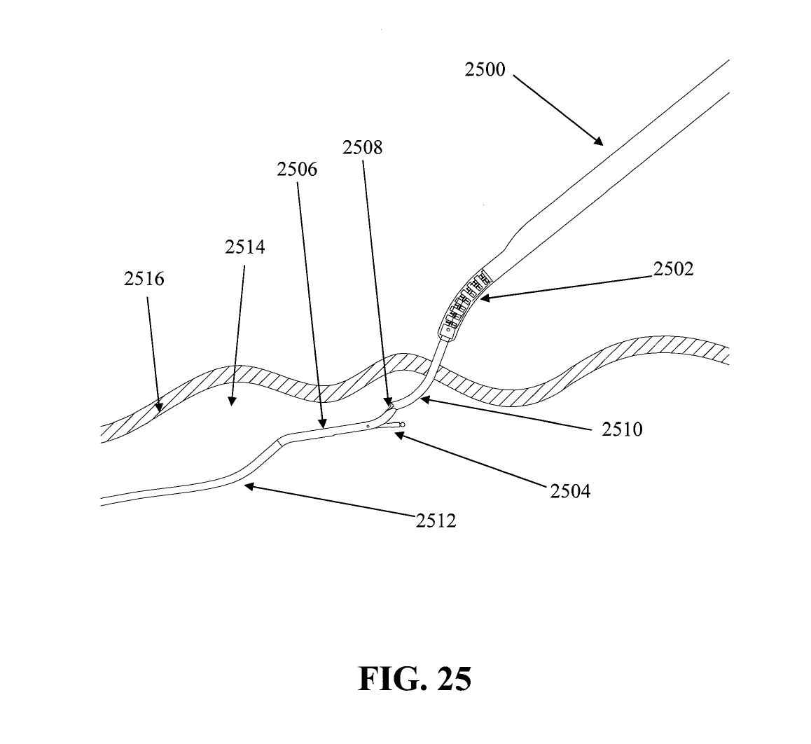

FIGS. 25-27 illustrate how the methods described here may be used to form tracts through one or more irregular tissue surfaces. This may be particularly desirable, because not all tissue surfaces are regular and not all tissue surfaces will coincide nicely with the shape or position of the tissue-locating member. Shown in FIG. 25 is distal portion of device (2500). Device (2500) may be similar to any of the devices previously described or incorporated by reference. Shown here is articulation section (2502), needle guide (2510), opening (2508), retainer (2504), tissue-locating member (2506), and guide cannula (2512). The function of each of these components has been described previously above.

In FIG. 25, an initial Seldinger stick has already been performed, and the device (2500) advanced so that the tissue-locating member (2506) and the opening (2508) reside within tissue lumen (2514). Here, the tissue has one or more irregular surfaces (2516). While the irregular surface of FIG. 25 is shown as having one or more undulations, the irregularity need not be so dramatic. Indeed, the irregularity may be one or more bends, curves, recesses, protrusions, any combination of these, or the like.

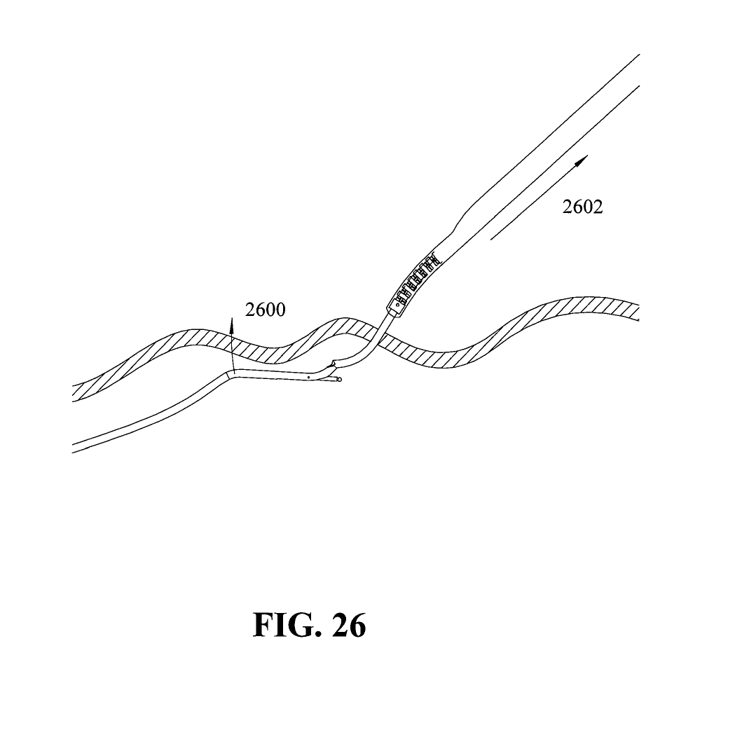

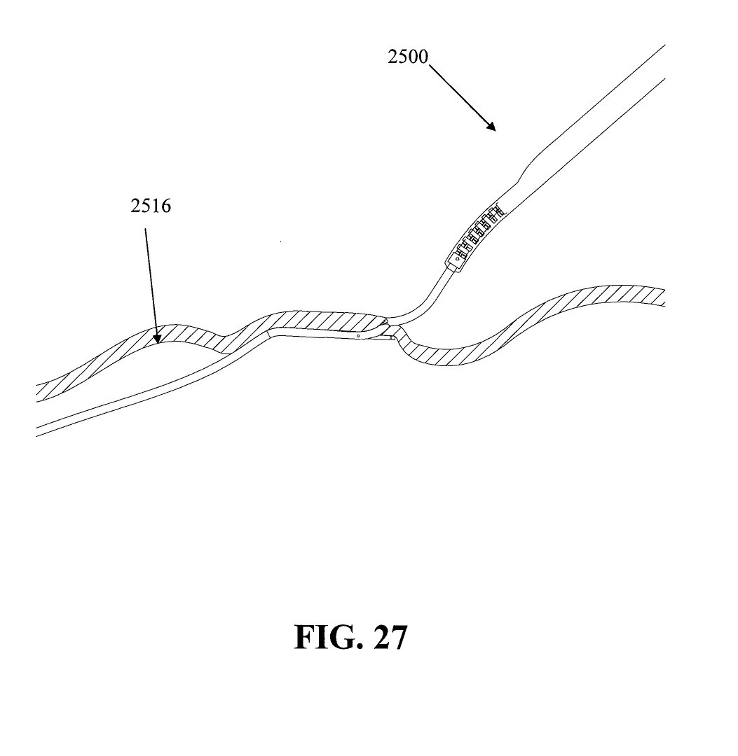

FIG. 26 shows the device after the articulation section (2502) has been released from an initial insertion position (shown in FIG. 25), so that the tissue-locating member may spring upward (as shown by arrow 2600). Also shown in FIG. 26, is how the device may be pulled proximally (shown by arrow 2602) so that the tissue locating member (2506) may be brought into contact with an inner surface of the tissue wall, as shown most clearly in FIG. 27. FIG. 27 shows device (2500) positioned so that a tissue-piercing member (not shown) may be advanced into the tissue. As shown there, a portion of tissue surface (2516) has effectively been straightened or stretched so that advancement of a tissue-piercing member therethrough may be accomplished with more ease.

### II. Examples

### A. Use of the Methods for Vascular Access (Arterial Punctures)

Arterial punctures were performed in 28 patients using the device shown in FIG. 4 with the following procedure. First, a baseline ultrasound scan was performed to assess vessel diameter and media thickness. The tissue-piercing member was then flushed with sterile heparinized saline via the plunger until the solution exited the distal end of the device and the flash port. A 21 gauge needle (0.14″ guide wire compatible) was then introduced into the common femoral artery using a standard Seldinger percutaneous technique. A 0.14″ guide wire than introduced through the needle and into the artery. The needle was then removed. The device of FIG. 4 was then advanced over the guidewire.

When the exit port of the guide cannula reached the skin surface, the guidewire was removed. The device was then rotated ¼ turn to the right or left to ease the advancement of the tissue-locating member into the artery. The device was then advanced further into the vessel, until a blood flash was observed from the marker port. The actuator was then engaged (to deploy a retainer) and gentle traction was applied to the handle until blood flash from the marker port shut off, indicating that the tissue-locating member was properly positioned against the artery wall. The plunger was then advanced to its stop, and the lever was actuated. The stop was then removed and the plunger was advanced to its maximum distance. A flash of blood was observed out of the plunger proximal opening, indicating that the tissue-piercing member had entered the lumen. The 0.14″ guide wire was then advanced through the plunger and tissue-piercing member into the artery lumen. The plunger port was then retracted. The retainer was then released, and the device was removed. A 6FR procedural sheath was then advanced over the 0.14″ guidewire. Contrast injection under fluoroscopy was then optionally performed to visualize the insertion site, followed by an inspection for damage. A percutaneous procedure was then performed. After the procedure, pressure was held at the sheath insertion site and the procedural sheath was removed. Pressure was held for 1 minute. After 1 minute, pressure was released and the site was inspected for signs of arterial bleeding. If bleeding was noted, pressure was held for an additional 1 minute and the site was then inspected for bleeding again. This continued in 1 minute pressure intervals.

The following results were obtained:

| Patient | | | | Time to Hemostasis |
|---|---|---|---|---|
| Number | Gender | Age | Disease | (min) |
| 1 | M | 50 | — | 3 |
| 2 | M | 68 | Slight Calcification | Tissue-piercing member |
| | | | | remained in artery wall* |
| 3 | M | 59 | — | 3 |
| 4 | M | 56 | | Tissue-piercing member |
| | | | | remained in artery wall* |
| 5 | M | 70 | Moderate | 4 |
| | | | Calcification | |
| 6 | M | 64 | Posterior and | 3 |
| | | | Lateral Plaques | |
| 7 | M | 55 | — | 4 |
| 8 | M | 71 | — | 12** |
| 9 | M | 88 | — | 7** |
| 10 | F | 47 | — | Tissue-piercing member |
| | | | | remained in artery wall* |
| 11 | M | 53 | — | 2 |
| 12 | M | 51 | — | 2 |
| 13 | M | 62 | Mild Calcification | 3 |
| 14 | F | 53 | — | 2 |
| 15 | F | 53 | Moderate | 3 |
| | | | Calcification | |
| 16 | M | 54 | — | 3 |
| 17 | M | 42 | Slight Calcification | 8 |
| 18 | M | 70 | Thick Plaque | 1 |
| 19 | F | 54 | Posterior Plaque | Tissue-piercing member |
| | | | | remained in artery wall* |
| 20 | F | 58 | Small Posterior | 3 |
| | | | Plaque | |
| 21 | F | 59 | — | 2 |
| 22 | F | 77 | Small Posterior and | 1 |
| | | | Anterior Plaques | |
| 23 | F | 52 | — | 2 |
| 24 | M | 70 | Thick Plaque | 2 |
| 25 | M | 62 | Large Posterior | 3 |
| | | | Plaque Mild | |
| | | | Calcification | |
| 26 | M | 78 | Diffuse Plaque | 1 |
| | | | Severe Calcification | |
| 27 | F | 56 | Posterior Plaque | Tissue-piercing member |
| | | | | remained in artery wall* |
| 28 | F | 69 | — | 6.3 |

*It is thought that in these patients, the tissue-piercing member was not sufficiently redirected due to operator error or inexperience, the length of the tissue-locating member (e.g., if this member were overly long, it would abut the lumen floor during the tissue deformation step), or the flexibility of the tissue-piercing member.
**ACT (activated clotting time) was greater than 500 min in these patients. Despite these patients being highly anti-coagulated, hemostasis was obtained in a relatively short amount of times.

Significantly, there were no significant adverse events or complications (including intimal dissection, acute vessel closure, thrombosis, retroperitoneal hemorrhage, thickening of the perivascular tissues, neural damage, infection, venous thrombosis, or pericatheter clot) and diagnostic or interventional procedures were successfully performed on all patients.

### B. Use of the Methods with Organs of the Cardiovascular System

An excised pig heart having generally irregular surfaces was fitted with a hose connection and pressurized with water to 80 mmHg using a hand-pumped garden sprayer with an on/off valve and a pressure gauge from a blood pressure arm cuff. A biopsy needle that accommodated a 0.035″ guidewire was used to perform the initial needle stick. A 0.035″ guidewire was introduced through the needle and the needle was then removed. A 7 F catheter that accepts the 0.035″ guidewire was advanced and inserted into the tissue. The 7 F catheter and the 0.035″ guidewire were then removed and the path was checked for leakage, where it was noted that leakage occurred. The septum was identified and the needle was inserted from the epicardium through the septum and into the right ventricle, forming an angled tract in tissue as described above. The guidewire was passed through the tricuspid valve and into the superior vena cava (which was fitted with a silicone tube). A 7 F catheter with a tapered tip was then inserted and removed. No noticeable leakage occurred, indicating that the tract had sealed almost instantaneously. The steps were essentially repeated, through a free wall of a left ventricle. The straight through tract leaked, while the angled tract did not.

### C. Use of the Methods with Organs of the Digestive System

An intact pig stomach was pressurized using a pressurized water canister with an on/off valve connected to, the esophagus. Pressure was monitored with a blood pressure cuff gauge (fitted to the small intestine). A hose extension was passed through the stomach to bypass the pyloric sphincter in order to ensure an accurate pressure reading. The pressure ranged between 4-6 mm Hg based on water filling stomach with no additional pressurization, to 8 mm Hg. A biopsy needle was advanced to create a shallow angled needle stick along the greater curvature of the stomach. A 0.035″ guidewire was introduced into the needle, and the needle was then removed, leaving the guidewire in place. A 7 F catheter was inserted over the guidewire and advanced across the stomach wall to form an angled tract as described above. The stomach tissue had an irregular and tough surface and the catheter had to be rotated to advance the catheter through the stomach wall. The catheter and guidewire were removed, and no leakage was observed, indicating the tract sealed almost instantaneously. The pressure was then increased to 60 mm Hg by pressing firmly on the stomach with a hand, and still there was no visible leak. The steps were then repeated with a 16 F catheter and no leakage was observed at 6 mm Hg, 20 mm Hg, or 60 mm Hg. A straight through tract was made using a needle accommodating a 0.035″ guidewire as described in the example just above, and significant leakage was noted.

1. A method for forming a tract in a tissue comprising:
a. advancing a tissue-locating member adjacent to a tissue wall;
b. deforming at least a portion of the tissue wall with the tissue-locating member, causing the portion to assume a tented configuration wherein the portion does not become folded; and
c. advancing a tissue-piercing member through the tented configuration to form the tract, wherein the tract provides access for one or more tools.
